(19) United States
(12) Patent Application Publication
(10) Pub. No.: US 2008/0027285 A1
(43) Pub. Date: Jan. 31, 2008

(54) Bending mechanism

(75) Inventor: Shinji Yasunaga, Asaka-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/880,407

(22) Filed: Jul. 20, 2007

(30) Foreign Application Priority Data
Jul. 21, 2006 (JP) JP 2006-199301

(51) Int. Cl. A61B 1/008

(52) U.S. Cl. 600/150

(57) ABSTRACT

A first bending portion, a second bending portion, a third bending portion, and a fourth bending portion are formed by a coiled spring. A front link member is disposed toward a front tube of the first bending portion, and a rear link member is disposed toward a rear tube of the fourth bending portion. A first intermediate link member is disposed between the first bending portion and the second bending portion, a second intermediate link member is disposed between the second bending portion and the third bending portion, and a third intermediate link member is disposed between the third bending portion and the fourth bending portion. A first actuator includes a tube member and an SMA wire member, and when a distance between the front link member and the tube member is shortened by driving the first actuator, the first bending portion is bent.

### CROSS-REFERENCE TO RELATED APPLICATION

The present application is based upon and claims the benefit of priority from the prior Japanese Patent Application No. 2006-199301 filed on Jul. 21, 2006; the entire contents of which are incorporated herein by reference.

### BACKGROUND OF THE INVENTION

1. Field of the Invention

The present invention relates to a bending mechanism, and in particular, to a bending mechanism which has a small diameter, and is capable of shortening a bending length by, such as a bending mechanism which is applicable as a bending mechanism for a medical catheter and an endoscope.

2. Description of the Related Art

So far, it is usual that a catheter which is used in combination with an endoscope does not have an active bending mechanism. However, in a case of using in a part in which, it is difficult to be inserted, the active bending mechanism may be useful as it improves insertability. Moreover, generally, although a pulling by a wire is used in the bending mechanism of the endoscope, an attempt has been made to pull electrically from a control point of view. Such a technology has been disclosed in Japanese Patent Application Laid-open Publication No. Hei 10-216238, and this has been described in FIG. 6A and FIG. 6B.

FIG. 6A and FIG. 6B show a shape memory alloy actuator in which a shape memory alloy is used. In the shape memory alloy actuator, as shown in the diagram, one end of a thread member 3 is fixed to one end portion of a curved part 2 of a long object 1, and an actuator 4 which is operated such that a length of the thread member 3 is changed is installed at the other end portion of the curved part 2. The actuator 4 has a guide member 5 and a shape memory member 10, and the guide member 5 is fixed along an axial direction of the long object 1. The guide member 5 guides an operation of the shape memory member 10, and the shape memory member 10 is elongated along the guide member 5 when the shape memory member 10 regains its state from a deformed state. A first end portion 8a of the shape memory member 10 is immovable with respect to the guide member 5, a second end portion 8b of the shape memory member 10 is movable, and the bending mechanism is structured by connecting the other end of the thread member 3 to the second end portion 8b of the shape memory member 10.

In the above mentioned conventional technology, since it is necessary to accommodate the thread member 3 and the shape memory alloy coil which is the shape memory member 10, inside the guide member 5, there are limitations on making a small diameter, and it is difficult to use in a catheter of a small diameter. Moreover, for bending the curved portion 2, a side of the long object 1 toward the guide member 5 has to be contracted, and a side opposite to the guide member 5 has to be elongated. However, when a bending length is made short, there is a lack of a generative force in the shape memory alloy coil due to an increase in a distortion with respect to the long object 1, and it is necessary to increase a length of the curved part 2.

Particularly, basically in one shape memory alloy actuator, since it is not possible to bend except in one direction, for bending in two directions, it is necessary to have two shape memory alloy actuators, and to secure a substantial bending length for bending in two directions. Thus, in a conventional method, it has been difficult to realize an active bending mechanism with a small diameter, and a short bending length.

### SUMMARY OF THE INVENTION

The present invention is made in view of the above mentioned circumstances, and an object of the present invention is to provide a bending mechanism with a small diameter and a short bending length, and having a simple and a low cost structure.

To solve the abovementioned problems, and to achieve the object, the inventors of the present invention provided a bending mechanism including

a coiled spring, and

an actuator in which, a wire member which is disposed along an axial direction of the coiled spring, and of which, an elongation and contraction is controllable is interpolated (inserted) into a tube member which can be bent, and one end of the wire member and one end of the tube member are joined.

The other end of the tube member is fixed to a first part of the coiled spring, or a member which is joined to the first part, and the other end of the wire member is joined to a second part of the coiled member which is at a position different from a position of the first part, in the axial direction of the coiled member, or a member which is joined to the first part.

The coiled spring is bent by changing a gap between the first part and the second part, with the elongation and contraction of the wire member.

Moreover, according a preferable aspect of the present invention, it is desirable that a plurality of actuators are disposed along the coiled spring, and a part to which the other end of the wire member and the other end of the tube member of each actuator are fixed or joined is at a different position in a circumferential direction of the coiled spring.

According to another preferable aspect of the present invention, it is desirable that a part to which, the other end of the wire member and the other end of the tube member of the plurality of actuators are joined or coupled is near an inside of a surface perpendicular to an axis of the coiled spring.

Moreover, according to still another preferable aspect of the present invention, it is desirable that the wire member is a shape memory alloy, and has a heating means which is capable of heating the shape memory alloy, and an elongation and a contraction of the shape memory alloy is controlled by controlling a temperature thereof.

### BRIEF DESCRIPTION OF THE DRAWINGS

FIG. 1 is a perspective view of a bending mechanism according to a first embodiment of the present invention;

FIG. 2 is an enlarged view of a first bending portion which forms the bending mechanism of the first embodiment;

FIG. 3 is a diagram for describing an actuator according to the first embodiment;

FIG. 4 is a diagram for describing a bending mechanism according to a second embodiment of the present invention;

FIG. 5 is an enlarged view of an area near a front end portion of a bending mechanism of a third embodiment of the present invention;

FIG. 6A is a diagram for describing a conventional bending mechanism; and

FIG. 6B is a diagram for describing the conventional bending mechanism.

### DETAILED DESCRIPTION OF THE INVENTION

Embodiments of a bending mechanism according to the present invention will be described below in detail by referring to the accompanying diagrams. However, the present invention is not restricted to the embodiments described below.

### First Embodiment

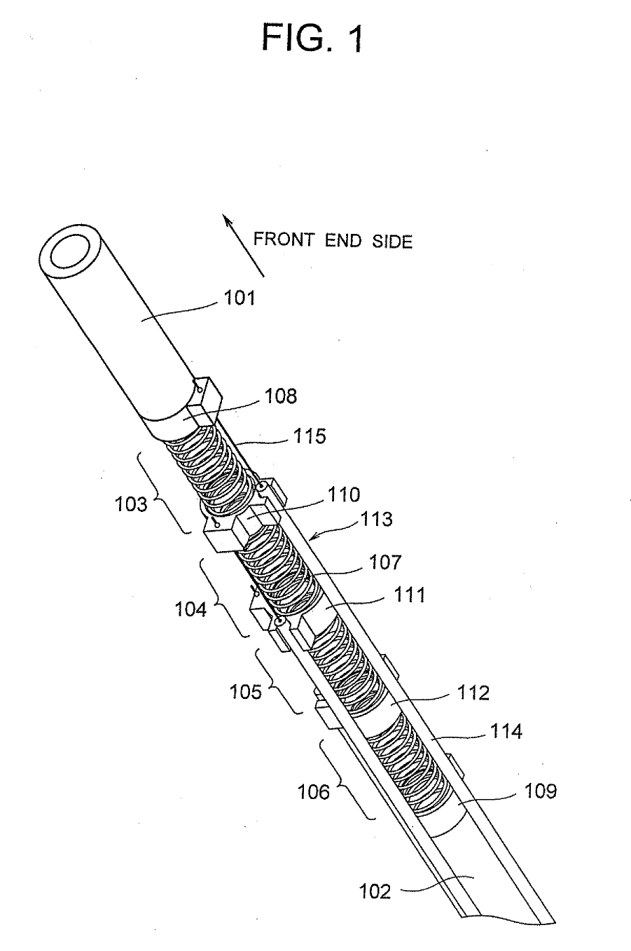

A first embodiment of the present invention will be described by using FIG. 1 to FIG. 3. FIG. 1 shows a perspective view of a bending mechanism of the first embodiment. A first bending portion 103, a second bending portion 104, a third bending portion 105, and a fourth bending portion 106 are disposed between a front tube 101 and a rear tube 102. The first bending portion 103 to the fourth bending portions 106 are formed by one integrated coiled spring 107. A front link member 108 is disposed toward the front tube 101 of the first bending portion 103, and a rear link member 109 is disposed toward the rear tube 102 of the fourth bending portion 106. A first intermediate link member 110 is disposed between the first bending portion 103 and the second bending portion 104, a second intermediate link member 111 is disposed between the second bending portion 104 and the third bending portion 105, and a third intermediate link member 112 is disposed between the third bending portion 105 and the fourth bending portion 106.

When the bending mechanism of the first embodiment is to be used for bending a front-end portion of a catheter, the rear tube is long and has a flexible structure, and according to the requirement, it is possible to adhere a flexible tube to a bending mechanism portion, as a covering.

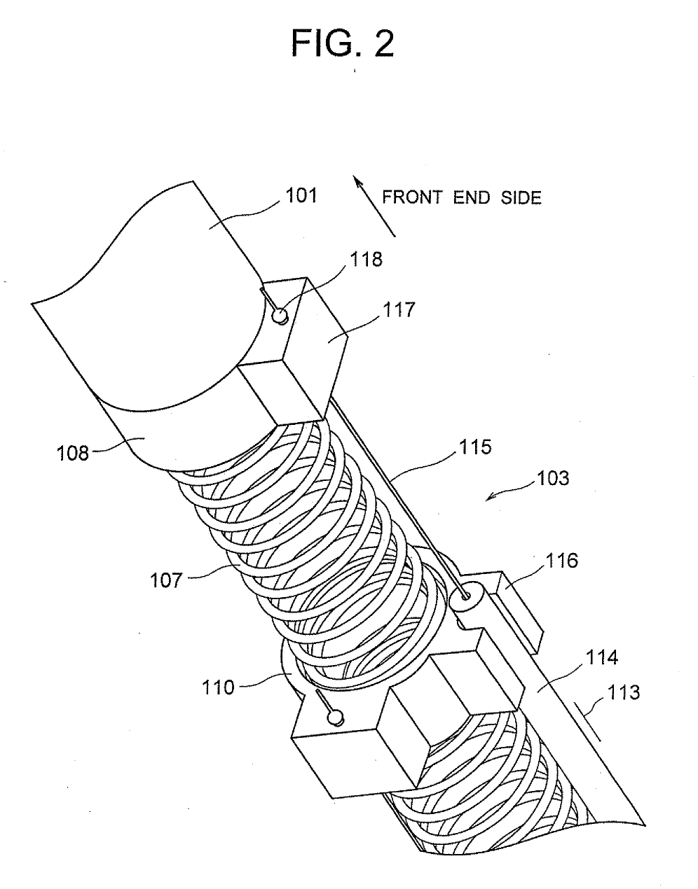

In a manipulator of the first embodiment, each of the first bending portion 103 and the fourth bending portion 106 can be driven independently. Here, an enlarged view of the first bending portion 103 is shown in FIG. 2, and an operation of the first bending portion 103 is described by using FIG. 2.

A first actuator 113 for bending is assembled in the first bending portion 103. The first actuator 113 includes a tube member 114 which is non-conductive, and a shape memory alloy wire (herein after called as SMA wire) 115 which is interpolated into the tube member 114, and a front end portion of the tube member 114 is fixed to a tube installing portion 116 of the first intermediate link member 110, by adhering etc. Moreover, a front end of the SMA wire 115 is inserted into a through hole which is formed in a protrusion 117 of the front link member 108, and a caulking member 118 is installed at a front-end side ahead of a through part. Here, a diameter of the caulking member 118 is let to be more than a diameter of the through hole in the projection 117.

Here, when the first actuator 113 is driven, and a distance between the caulking member 118 and the tube member 114 becomes short, a coiled spring 107 between the front link member 108 and the first intermediate link member 110 is pulled on an opposite side of the caulking member 118 and the tube member 114, sandwiching a center of the coiled spring 107 in an axial direction (longitudinal direction). As a result, the coiled spring 107 is bent in a direction of protruding portion of the protrusion 117 with an area near the tube installing portion 116 as a base point.

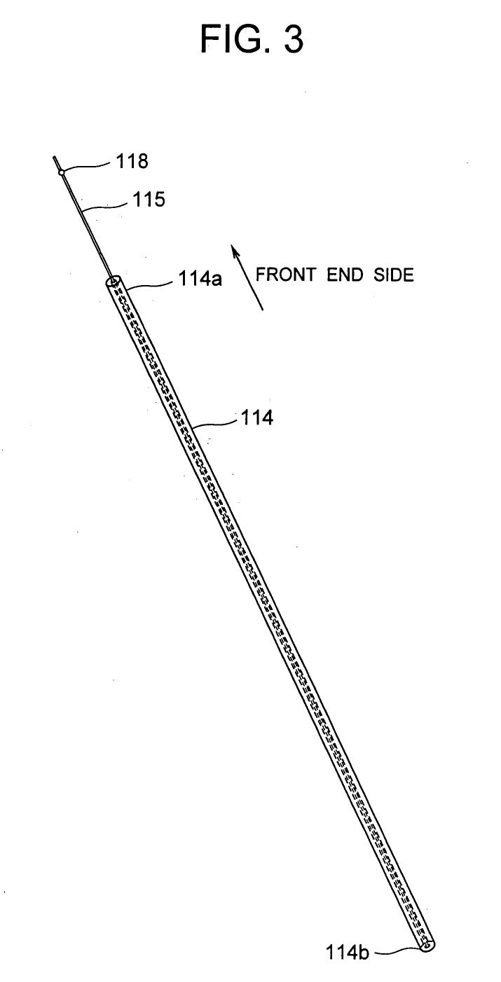

Next, an operation of the first actuator 113 will be described by using FIG. 3. The SMA wire 115 inserted into the tube member 114 is slidable at a front-end side of the tube member 114, but is fixed at a rear-end portion 114b of the tube member 114.

Here, although it is not shown particularly in a diagram, a wire of a small diameter is fixed to both ends of the SMA wire 115, and when heated by a predetermined current which is allowed to flow, the SMA wire 115 is contracted, and a distance between the caulking member 118 installed near the front end of the SMA wire 115, and a front end portion 114a of the tube member 114 fixed to the tube installing portion 118 becomes short. In an actuator having such structure, since it is possible to increase a length of the SMA wire 115 rather than the distance between the front end portion 114a of the tube member 114 and the caulking member 118 which are practically displaced, it is possible to achieve a sufficiently substantial displacement. Moreover, as an actuator, the structure is simple with the SMA wire 115 passed through the tube member 114, it is possible to generate a substantial force with very small diameter.

Therefore, when the current is applied through a wire having a small diameter, which is fixed to the both ends of the SMA wire 115, the first bending portion 103 is bent in a direction of the projection 117 and the tube installing portion 116 with the center of the coiled spring 107 in the axial direction (longitudinal direction) as a neutral axis, and when the current passed through the wire having the small diameter which is fixed to the both ends of the SMA wire 115, is stopped, the SMA wire 115 is pulled due to an elasticity of the coiled spring 107, and as a result, the first bending portion 103 takes a form of a straight line. Moreover, by controlling the current passed through the SMA wire 115 to a proper value, it is also possible to control the first bending portion 103 to a predetermined angle of bending.

The second bending portion 104, the third bending portion 105, and the fourth bending portion 106 also have the same structure, and an actuator having a same structure as the actuator 103 is disposed to each of the second bending portion 104, the third bending portion 105, and the fourth bending portion 106. Here, since the four actuators are disposed at positions differing by 90 degrees in a circumferential direction of the coiled spring 107, by controlling each of the actuators independently, it is possible to bend the front tube 101 with respect to the rear tube 102, in an arbitrary direction.

In this manner, in the bending mechanism of the first embodiment, the bending portion is formed by the coiled spring, and the actuator having a very small diameter and capable of achieving a sufficient amount of displacement is disposed on a circumference thereof. As a result, it is possible to provide the bending mechanism having a small diameter and a small bending length as a structure which is easy to manufacture at a low cost. Furthermore, as the operation of each of the four bending portions is completely independent, it has an advantage of having an easy control. Moreover, by causing the four actuators to contract simultaneously, it is also possible to displace the front tube 101 in the axial direction of the coiled spring 107, with a direction of the front tube 101 maintained to be almost parallel with respect to the rear tube 102. Such an operation is difficult in a bending mechanism in which an ordinary angle wire is used.

### Second Embodiment

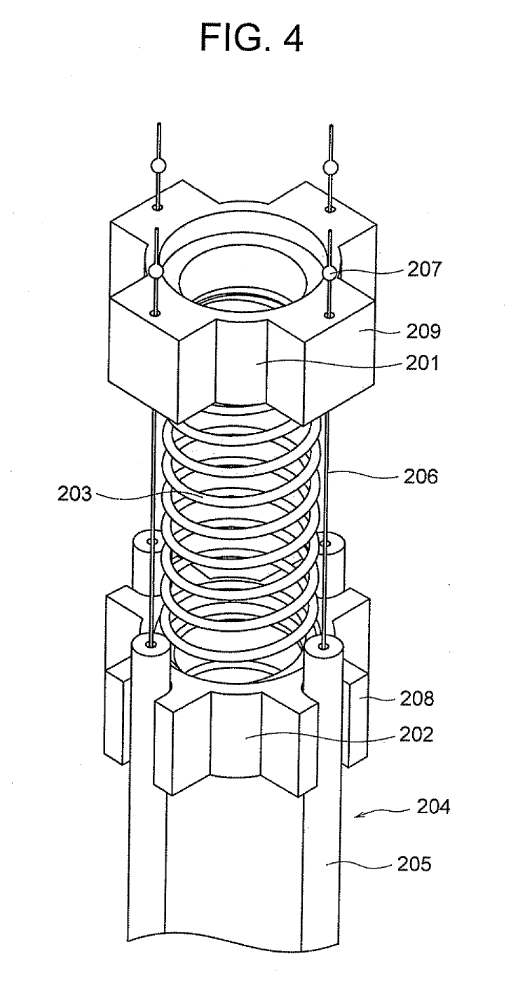

A second embodiment of the present invention will be described below by using FIG. 4. An overall structure of a bending mechanism of the second embodiment is similar to the structure of the bending mechanism described in the first embodiment, but a portion shown in FIG. 4 is different. As shown in FIG. 4, a bending portion positioned between a front link member 201 and a rear link portion 202 is formed by a coiled spring 203, and an actuator 204 is disposed at an outer circumference thereof.

The actuator 204 according to the second embodiment includes a tube member 205, and an SMA wire 206 which is inserted into the tube member 205, and a front end portion of the tube member is fixed at a tube installing portion 208 of the rear link member 202, by adhering etc. Moreover, a front end of the SMA wire 206 is inserted into a through hole which is formed in a protrusion 209 of the front link member 201, and a caulking member 207 is installed at a front side ahead of a through part. Here, a diameter of the caulking member 207 is let to be more than a diameter of the through hole in the projection 117.

Here, when the actuator 204 is driven, and a distance between the caulking member 207 and the tuber member 205 becomes short, a coiled spring 203 between the front link member 201 and the rear link member 202 is pulled on an opposite side of the caulking member 207 and the tube member 205, sandwiching a center of the coiled spring 203 in an axial direction (longitudinal direction). As a result, the coiled spring 203 is bent in a direction of protruding portion of the protrusion 117, with an area near the tube installing portion 208 as a base point.

Here, although only one actuator is described, and a reference numeral is assigned to this actuator, four actuators can be disposed at positions differing by 90 degrees in a circumferential direction of the coiled spring 203, between the front link member 201 and the rear link member 202 as it is revealed from FIG. 4. It is possible to drive each of these four actuators independently, and by driving a specific actuator, it is possible to bend the front link member 201 with respect to the rear link member 202, in a direction in which the actuator is disposed, with the center of the coiled spring 203 in the axial direction as a neutral axis. Moreover, by controlling to two adjacent actuators to a predetermined amount of displacement, it is possible to bend in an arbitrary direction. Furthermore, by driving the four actuators simultaneously, it is possible to displace the front link member 201 with respect to the rear link member 202 in the axial direction of the coiled spring 203.

In this manner, in the bending mechanism of the second embodiment, a plurality of actuators are disposed in which the bending portion is formed by a coiled spring, and an outer circumference thereof is very small, and in which a sufficient amount of displacement is achieved. As a result, it is possible to provide the bending mechanism having a small diameter and a small bending length as a structure which is easy to manufacture at a low cost. Moreover, by causing the four actuators to contract simultaneously, it is also possible to displace in an axial direction of the bending portion. Such an operation is difficult in a bending mechanism in which an ordinary angle wire is used.

### Third Embodiment

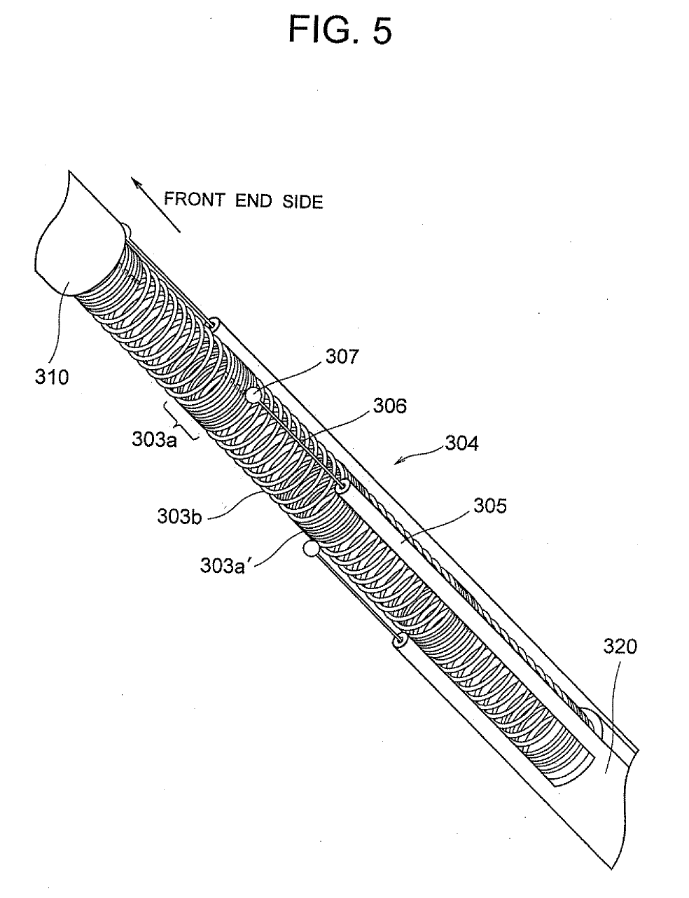

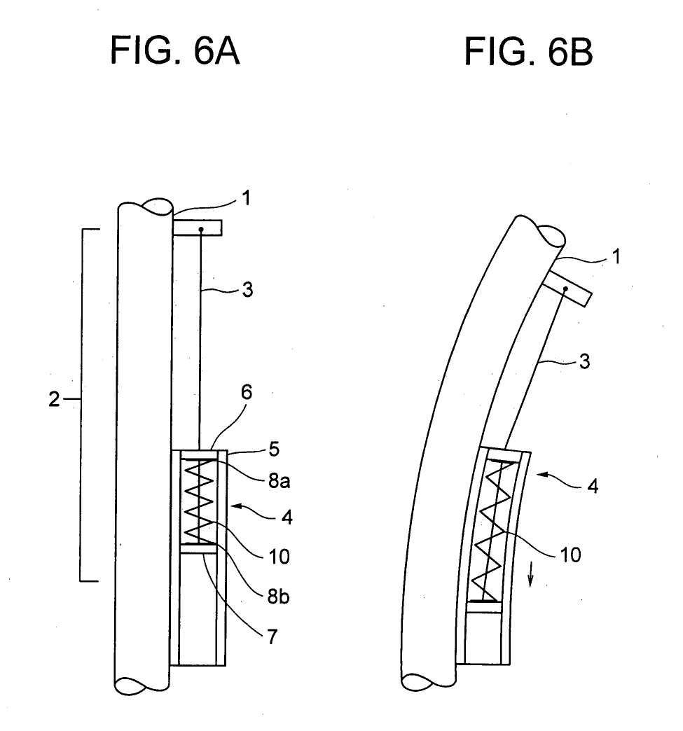

A third embodiment of the present invention will be described below by using FIG. 5.

A bending mechanism according to the third embodiment, as shown in FIG. 5, has a coiled spring 303 installed between a front tube 310 and a rear tube 320, which includes a finely spaced portion 303a having a comparatively large number of turns per a unit length in an axial direction (in other words, a comparatively small spring pitch), and a coarsely spaced portion 303b having a comparatively small number of turns per unit length in the axial direction (in other words, a comparatively large spring pitch), arranged alternately, and an actuator 304 is disposed at an outer circumference of the coiled spring 303. It is possible to structure the finely spaced portion 303a such that each winding makes a close contact mutually till a predetermined load is exerted.

The actuator 304 according to the third embodiment includes a tube member 305, and an SMA wire 306 which is inserted into the tube member 305. A front end portion of the tube member 305 is fixed to a finely spaced portion 303a′ by adhering etc., and a front end of the SMA wire 306 is fixed to at least one winding belonging to the finely spaced portion 303a, by a caulking member 307.

In other words, in the third embodiment, the structure is such that each link member described in the first embodiment and the second embodiment is omitted, and instead of these link members, finely spaced portions (303a and 303a′ in FIG. 5) provided to the coiled spring are used.

When the actuator 304 is driven, and a distance between the caulking member 307 and the tube member 305 becomes short, a coarsely spaced portion 303b of a coiled spring 303 between the finely spaced portion 303a and the finely spaced portion 303a′ is pulled at an opposite side of the caulking member 307 and the tube member 305, sandwiching a center of the coiled spring 303 in an axial direction (longitudinal direction). As a result, the coiled spring 303 is bent in a direction of the caulking member 307, with an area near an installing portion of the tube member 305 to the finely spaced portion 303a′ as a base point.

Here, although only one actuator is described, and a reference numeral is assigned to this actuator, it is possible to dispose four actuators at positions differing by 90 degrees in a circumferential direction of the coiled spring 303 for example, equipped with a plurality of finely spaced portions and coarsely spaced portions as revealed in FIG. 5. It is possible to drive each of these four actuators independently, and by driving a specific actuator, it is possible to bend in a direction in which the actuator is disposed, with the center of the coiled spring 303 in the axial direction as a neutral axis. Moreover, by controlling two adjacent actuators to a predetermined amount of displacement, it is possible to bend in an arbitrary direction. Furthermore, by driving the four actuators simultaneously, it is possible to displace the finely spaced portion 303a to displace with respect to the finely spaced portion 303a′, in the axial direction of the coiled spring 303.

In this manner, in the bending mechanism of the third embodiment, a plurality of actuators is disposed in which the bending portion is formed by a coiled spring, and an outer circumference thereof is very small, and in which a sufficient amount of displacement is achieved. As a result, it is possible to provide a bending mechanism having a small diameter and a small bending length as a structure which is easy to manufacture at a low cost. Moreover, by causing the four actuators to contract simultaneously, it is also possible to displace in an axial direction of the bending portion. Such an operation is difficult in a bending mechanism in which an ordinary angle wire is used.

In each of the abovementioned embodiments, the description is made by using a coiled spring. However, the present invention is not restricted to the coiled spring, and it is possible to use other elastic body which is made of a rubber having a predetermined restoring force, silicon, and other resins. When these materials are used, it is possible to let an elastic body having other appropriate cross-sectional shape such as a hollow circular column and an angular column. Moreover, in each of the abovementioned embodiments, the description is made by using a material which is contracted by heating, as a wire member which forms the actuator. However, the present invention is not restricted to a material of this type, and it is possible to use a material of a type which is elongated by heating, as a wire member.

Moreover, a case in which the present invention is applied to a catheter is taken into consideration. A catheter has a comparatively small number of built-in components inside. Therefore, it is preferable that at the time of inserting a catheter into a part in which it is difficult to be inserted, it is possible to shrink (shorten) a front end portion.

As it has been described above, the bending mechanism according to the present invention is useful as a bending mechanism in which a small diameter and a short bending length is sought, and is particularly suitable for a bending mechanism which is used in an endoscope etc.

The present invention shows an effect that it is possible to provide a bending mechanism having a small diameter and a short bending length, as a structure which is easy to manufacture at a low cost.

What is claimed is:

1. A bending mechanism comprising:
a coiled spring; and
an actuator in which, a wire member which is disposed along an axial direction of the coiled spring, and of which, an elongation and contraction is controllable, is inserted into a tube member which can be bent, and one end of the wire member and one end of the tube member are joined, wherein
the other end of the tube member is fixed to one of a first part of the coiled spring, or a member which is joined to the first part, and
the other end of the wire member is joined to one of a second part of the coiled member which is at a position different from a position of the first part, in the axial direction of the coiled member, or a member which is joined to the first part, and
the coiled spring is bent by changing a gap between the first part and the second part, with the elongation and contraction of the wire member.

2. The bending mechanism according to claim 1, wherein
a plurality of actuators are disposed along the coiled spring, and a part to which the other end of the wire member and the other end of the tube member of each actuator are fixed or joined is at a different position in a circumferential direction of the coiled spring.

3. The bending mechanism according to claim 2, wherein
a part to which, the other end of the wire member and the other end of the tube member of the plurality of actuators are joined or coupled is near an inside of a surface perpendicular to an axis of the coiled spring.

4. The bending mechanism according to claim 1, wherein
the wire member is a shape memory alloy, and has a heating means which is capable of heating the shape memory alloy, and an elongation and a contraction of the shape memory alloy is controlled by controlling a temperature thereof.
